(19) United States
(12) Patent Application Publication
(10) Pub. No.: US 2013/0226211 A1
(43) Pub. Date: Aug. 29, 2013

(54) Tattoo Needle Tip Equipped with Capillary Ink Reservoir, Tattoo Tube Having Handle and Said Tattoo Needle Tip, and Assembly of Said Tattoo Needle Tip and Tattoo Needle

(71) Applicant: Long XIAO, (US)

(72) Inventor: Long XIAO, TORONTO (CN)

(73) Assignee: Long XIAO, TORONTO (CA)

(21) Appl. No.: 13/773,616

(22) Filed: Feb. 21, 2013

(30) Foreign Application Priority Data
Feb. 23, 2012 (CN) 201220061094.X

(51) Int. Cl. A61M 37/00

(52) U.S. Cl. CPC A61M 37/0084; A61M 37/0076
USPC 606/186

(57) ABSTRACT

The present invention provides a tattoo needle tip equipped with a capillary ink reservoir, a tattoo tube having the said tattoo needle tip, and an assembly of the said tattoo needle tip and a tattoo needle. The tattoo needle tip equipped with a capillary ink reservoir comprises a needle tip body. A tattoo needle passage is provided inside the needle tip body. A needle tip opening is provided at one end of the needle tip body, and the needle tip opening and the tattoo needle passage are connected. The needle tip body is provided with at least one ink reservoir for containing and storing ink. The ink reservoir is provided with at least one ink chamber with capillary action. At least one ink guiding passage connects the capillary ink chamber with the needle tip opening. The tattoo needle tip equipped with an ink reservoir utilizes capillary action to store ink in the ink reservoir, thereby having a big capacity of ink storage and preventing ink leakage from easily occurring.

### RELATED APPLICATIONS

This application claims priority to Chinese Application Serial Number 201220061094.X, filed Feb. 23, 2012, which is herein incorporated by reference.

### FIELD OF THE INVENTION

The present invention relates to a device for tattooing or permanent make-up, particularly relates to a tattoo needle tip equipped with a capillary ink reservoir, a tattoo tube having a handle and said tattoo needle tip, and an assembly of said tattoo needle tip and a tattoo needle.

### BACKGROUND OF THE INVENTION

Conventional tattoo devices generally comprise a tattoo machine, a tattoo handle, a tattoo needle tip, and a tattoo needle. Users need to assemble these components before use. While assembling, the tattoo handle is connected with the tattoo machine, the tattoo needle tip is mounted to the handle, the tattoo needle passes through a central hole in the handle, and then the tip of the tattoo needle reaches the opening of the tattoo needle tip.

In prior art, the basic configuration of a tattoo needle tip is substantially a cylindrical tube. One end of the cylindrical tube is provided with a truncated cone part. The end head of the truncated cone part is provided with a tattoo needle passage and a needle tip opening for a tattoo needle to protrude.

The basic structure of a common tattoo needle comprises a needle head and a needle bar connecting with the needle head. The needle head usually is composed of a plurality of thin needle wires. A plurality of thin needle wires constitute a cylindrical shape to form a round needle head, or a plurality of thin needle wires are arranged side by side to form a flat needle head. A needle tip equipped with around or rhombus opening is used corresponding to the round needle head, and a needle tip equipped with a rectangle opening is used corresponding to the flat needle head. The needle tip equipped with a rectangle opening can also be designed to be open, that is, designed as a rectangle slot. According to different diameters of the round needle head and different widths of the flat needle head, the needle tip opening and tattoo needle passage are accordingly designed with corresponding sizes.

The truncated cone part of the needle tip is designed to form a truncated cone shaped inner cavity inside the needle tip, and the truncated cone shaped inner cavity is also named as an ink storage cavity. The internal wall from the truncated cone shaped inner cavity to the needle tip opening mostly is designed to be a streamlined smooth internal wail, so as to prevent the needle tip from being bent by the resistance caused during inserting the tattoo needle.

While working, the tattoo needle processes a high-frequency, linear reciprocating motion, so, the needle tip dipped with the ink rushes out of the needle tip opening to penetrate the skin, and then the needle tip returns to the needle tip opening to dip the ink to rush out again. The tattooist will dip the needle tip in a small ink cup to fill the needle tip with the ink; interstice is provided between the internal wall of the needle tip opening and the tattoo needle, so, usually the tattoo ink will be sucked into the needle tip opening, and the needle tip opening and the internal wall of the tattoo needle passage will be dipped with the tattoo ink. Some tattooists will start the tattoo machine for a short time while dipping the tattoo ink, so, the tattoo needle will process an up-and-down, reciprocating, high-speed motion, and the ink will quickly be raised to fill the inner cavity of the truncated cone part. While making tattoos, the smooth internal wall of the inner cavity of the tattoo needle tip is not good for holding the ink, and the ink will flow towards the needle tip opening by the action of gravity and the motion of the tattoo needle.

Currently, a general problem that occurs when a tattooists uses these needle tips is that, if the needle tip is filled with too much ink, the ink will easily leak to stain the tattoo design while making tattoos. To avoid the ink leakage, the tattooist has to add a small amount of ink each time, and increases the frequency of adding ink. During tattooing, adding ink frequently will bring trouble and inconvenience to the tattooists, so, break or insufficiency of ink supply will frequently occur during making tattoos. This will result in difficulties when making smooth, long tattoo lines for the tattooist.

### SUMMARY OF THE INVENTION

An object of the present invention is to provide a tattoo needle tip equipped with a capillary ink reservoir, which has a function of storing ink and holding ink and does not easily leak ink, so as to achieve that the ink dipped by the tattoo needle tip at one time can last the tattoo process for a comparatively long time, thereby preventing the tattooist from frequently dipping the ink while making tattoos.

Another object of the present invention is to provide a tattoo tube having a handle and a tattoo needle tip equipped with a capillary ink reservoir. It is convenient for tattoo artists to use.

The other object of the present invention is to provide an assembly of a said tattoo needle tip equipped with a capillary ink reservoir and a tattoo needle, which can achieve that ink dipped by the tattoo needle tip at one time can last the tattoo process for a comparatively long time, thereby preventing the tattooist from frequently dipping the ink while making tattoos.

To achieve the above-mentioned objects, a tattoo needle tip equipped with a capillary ink reservoir of the present invention comprises a needle tip body. A tattoo needle passage for mounting a tattoo needle is provided inside the needle tip body. A needle tip opening for the tip of the tattoo needle to protrude therefrom is provided at one end of the needle tip body. The needle tip opening and the tattoo needle passage are connected. The needle tip body is provided with at least one ink reservoir for containing and storing ink. The ink reservoir is provided with at least one ink chamber with capillary action. At least one ink guiding passage connects the ink chamber with the needle tip opening.

The ink chamber is a plurality of capillary grooves provided on the ink reservoir.

The ink reservoir is made from porous materials or fiber materials, and, the ink chamber is pores on the ink reservoir or interstices among fibers.

The ink reservoir is arranged on the needle tip body as one unit, or the ink reservoir is demountable to be assembled to the needle tip body. The ink guiding passage is arranged on the needle tip body between the ink reservoir and the tattoo needle passage or is formed by the tattoo needle passage.

A tattoo tube having a tattoo needle tip equipped with a capillary ink reservoir and a handle of the present invention comprises a handle, and a tattoo needle tip equipped with a capillary ink reservoir provided on the handle. The tattoo needle tip equipped with a capillary ink reservoir comprises a needle tip body. A tattoo needle passage for mounting a tattoo needle is provided inside the needle tip body. A needle tip opening for the tip of the tattoo needle to protrude therefrom is provided at one end of the needle tip body. The needle tip opening and the tattoo needle passage are connected. The needle tip body is provided with at least one ink reservoir for containing and storing ink. The ink reservoir is provided with at least one ink chamber with capillary action. At least one ink guiding passage connects the ink chamber with the needle tip opening.

The handle comprises a handle body. The handle body comprises a holding grip, and a handle neck provided at one end of the holding grip. The tattoo needle tip equipped with a capillary ink reservoir is connected with the other end of the holding grip as one unit. The handle body is provided with a guide hole that is connected with the tattoo needle passage by in turn perforating the inside of the handle neck and the holding grip.

The handle comprises a handle body. The handle body comprises a holding grip, a handle neck and a needle tip installation part. The handle neck and the needle tip installation part are respectively provided at the two opposite ends of the holding grip. The handle body is provided with a guide hole that is connected with the needle tip installation part by in turn perforating the inside of the handle neck and the holding grip. The tattoo needle tip equipped with a capillary ink reservoir is demountable to be assembled to the needle tip installation part.

The needle tip installation part is provided at the inner side of the opening of a guide hole at one end of the holding grip. The needle tip installation part is provided with a female connector. The outside of the end of the needle tip body away from the needle tip opening is provided with a male connector corresponding to the female connector. The male connector of the needle tip body is fastened in the female connector.

An assembly of a tattoo needle tip equipped with a capillary ink reservoir and a tattoo needle of the present invention comprises a tattoo needle tip equipped with a capillary ink reservoir and a tattoo needle assembled in the tattoo needle tip equipped with a capillary ink reservoir. The tattoo needle tip equipped with a capillary ink reservoir comprises a needle tip body. A tattoo needle passage is provided inside the needle tip body. A needle tip opening is provided at one end of the needle tip body, and the needle tip opening and the tattoo needle passage are connected. The needle tip body is provided with at least one ink reservoir for containing and storing ink. The ink reservoir is provided with at least one ink chamber with capillary action. At least one ink guiding passage connects the ink chamber with the needle tip opening. The tattoo needle is assembled in the tattoo needle passage of the needle tip body, and the tip of the tattoo needle is located at one end in the tattoo needle passage near the needle tip opening.

The tattoo needle comprises a needle head and a needle bar connecting with the needle head. The needle head is contained in the tattoo needle passage of the needle tip body. The tip of the needle head is located at one end in the tattoo needle passage near the needle tip opening. The needle bar protrudes out of the needle tip body from the end of the tattoo needle passage away from the needle tip opening, so as to connect the component for driving the tattoo needle.

The advantages of the present invention: the present invention utilizes capillary action to store ink in the ink reservoir, thereby having a big capacity of ink storage and ink leakage not easily occurring. The ink in the ink reservoir is continuously transported through the ink guiding passage to the needle tip opening, and the needle tip continuously dips the ink at the needle tip opening by the reciprocating motion of the tattoo needle. Thus, the ink dipped by the tattoo needle tip at one time can last the tattoo process for a comparatively long time, thereby preventing the tattooist from frequently dipping the ink while making tattoos, and saving the time of tattoo process.

The characteristic and the technical solution of the present invention are best understood from the following detailed description with reference to the accompanying figures, but the figures are only for reference and explaining, not to limit the scope of the invention.

### BRIEF DESCRIPTION OF THE DRAWINGS

The technical solution and the beneficial effects of the present invention are best understood from the following detailed description with reference to the accompanying figures and embodiments.

FIG. 1 is a perspective view of a tattoo needle tip equipped with a capillary ink reservoir in accordance with an embodiment of the present invention;

FIG. 2 is similar to FIG. 1 but with a portion cut out;

FIG. 3 is a perspective view of a tattoo tube having a tattoo needle tip equipped with a capillary ink reservoir and a handle in accordance with an embodiment of the present invention;

FIG. 4 is an exploded view of a tattoo tube having a tattoo needle tip equipped with a capillary ink reservoir and a handle in accordance with another embodiment of the present invention;

FIG. 5 is a perspective view of an assembly of a tattoo needle tip equipped with a capillary ink reservoir and a tattoo needle in accordance with an embodiment of the present invention;

FIG. 6 is similar to FIG. 5 but with a portion cut out;

FIG. 7 is a perspective view of a tattoo needle tip equipped with a capillary ink reservoir, a handle and a tattoo needle of the present invention.

### DESCRIPTION OF THE PREFERRED EMBODIMENTS

To further set forth the technical solution adopted by the present invention and the effect, the present invention is described in detail with reference to the preferred embodiments and the accompanying figures.

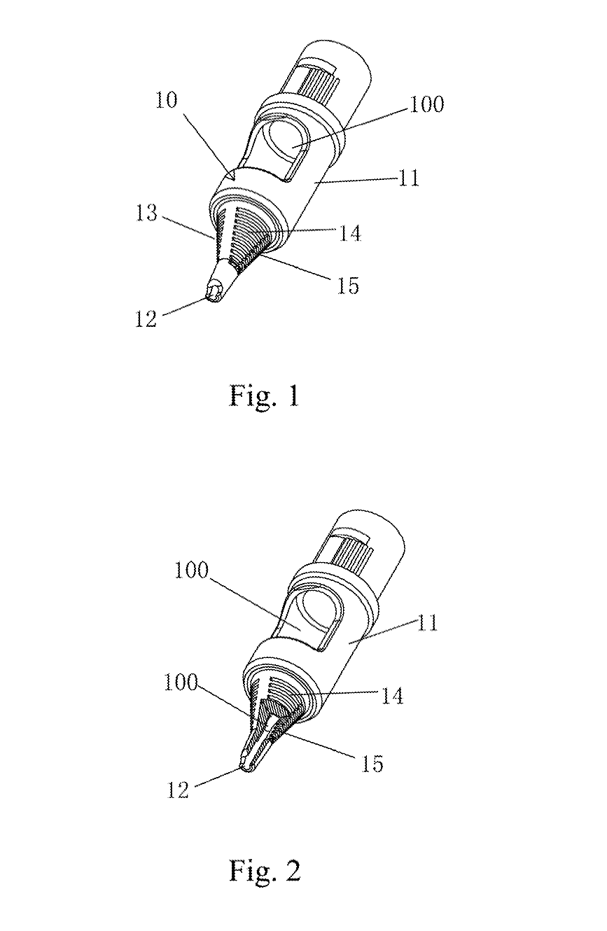

Referring to FIGS. 1-2, a tattoo needle tip 10 with a capillary ink reservoir in accordance with an embodiment of the present invention comprises a needle tip body 11. A tattoo needle passage 100 for mounting a tattoo needle is provided inside the needle tip body 11. A needle tip opening 12 is provided at one end of the needle tip body 11. The needle tip opening 12 and tattoo needle passage 100 are connected. The tip of the tattoo needle will protrude out of the needle tip opening 12. The needle tip body 11 is provided with at least one ink reservoir 13 for containing and storing ink. The ink reservoir 13 is provided with at least one ink chamber 14 with capillary action. At least one ink guiding passage 15 connects the ink chamber 14 with the needle tip opening 12. Tattoo ink can be contained and stored in the capillary ink chamber 14 of the ink reservoir 13, and can flow through the ink guiding passage 15 to the needle tip opening 12, thereby being dipped by a tattoo needle to make tattoos.

The ink reservoir 13 and the needle tip body 11 are designed as one unit, or the ink reservoir 13 is demountable to be assembled to the needle tip body 11. The ink chamber 14 can be a plurality of spaced capillary grooves provided on the ink reservoir 13. The interstice in the capillary groove is a capillary interstice, which has capillary action, and the ink reservoir adsorbs ink through the capillary action. The ink reservoir 13 can be made from porous materials, that is, the ink chamber 14 is pores on the ink reservoir 13, and the pores are capillary pores with capillary action. Or, the ink reservoir 13 is made from fiber materials, such as a plurality of fibers. The interstices among the fibers of the ink reservoir 13 form the ink chamber 14, and the interstices are capillary interstice also with capillary action. The present invention adopts the above-mentioned capillary ink reservoir 13 to store and supply ink, thereby having big capacity of ink storage and preventing ink leakage, comparing with the ink storage of the cavity with smooth internal wall adopted in the prior art. The ink reservoir 13 can be arranged at any part of the needle tip body 11 near the needle tip opening 12. The ink guiding passage 15 can be arranged on the needle tip body 11 between the ink reservoir 13 and the tattoo needle passage 100, such as on the wail of the needle tip body 11, the end of the needle tip body 11 near the needle tip opening 12 and so on. The ink reservoir 13 can be made as one unit with the needle tip body 11, thereby being connected with the needle tip body 11 as one unit. Or, the ink reservoir 13 can also be respectively made and then be connected with the needle tip body 11. The ink guiding passage 15 is a capillary slot that can guide ink to the needle tip opening 12, whereby ink leakage does not easily occur. The ink guiding passage 15 can be arranged on the needle tip body 11 near the position of the ink reservoir 13, which connects with the ink chamber 14 and the needle tip opening 12 via a section of tattoo needle passage 100, so as to be capable of guiding and transporting the ink stored in the ink chamber 14 to the needle tip opening 12. The ink guiding passage 15 can also be formed by the tattoo needle passage 100. When the ink chamber 14 of the ink reservoir 13 connects with the tattoo needle passage 100, the tattoo needle passage 100 can also be used as the ink guiding passage. After a tattoo needle is assembled to the tattoo needle passage 100, the ink stored in the ink chamber 14 can flow to the needle tip opening 12 along the gap between the tattoo needle and the tattoo needle passage 100, for the tip of the tattoo needle to dip, whereby no additional ink guiding passage is required by the ink reservoir 13.

Referring to FIGS. 1-2, in the present embodiment, the ink reservoir 13 is arranged at the end of the needle tip body 11 near the needle tip opening 12. The end of the needle tip body 11 is conical, or cylindrical. The ink chamber 14 is capillary grooves provided on the ink reservoir 13. The ink reservoir 13 is provided with at least one ink guiding passage 15 connecting with the ink chamber 14 and the needle tip opening 12.

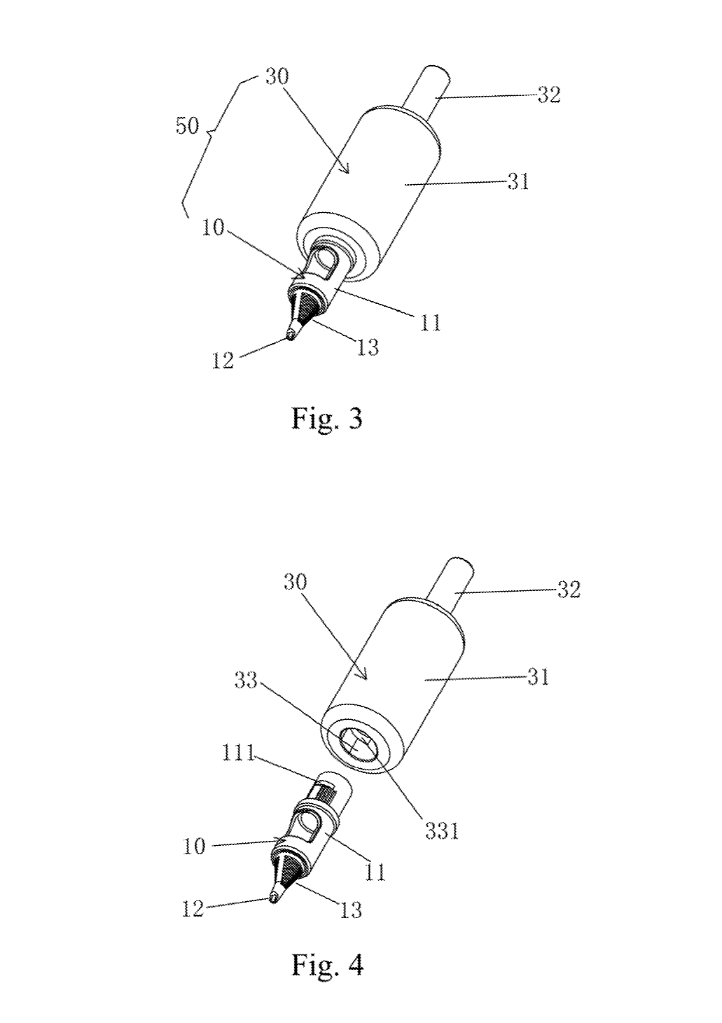

Referring to FIGS. 3-4, a tattoo tube 50 having a tattoo needle tip equipped with a capillary ink reservoir and a handle of the present invention comprises: a handle 30, and a tattoo needle tip provided on the handle 30. The tattoo needle tip is configured as the above-mentioned tattoo needle tip 10 with a capillary ink reservoir. The handle 30 is demountable to be assembled to the tattoo needle tip 10 with a capillary ink reservoir, or is made as one unit with the tattoo needle tip 10 equipped with a capillary ink reservoir. The assembly of a tattoo needle tip equipped with a capillary ink reservoir and a handle forms a tattoo tube 50, a component of a tattoo device. A tattoo artist can assemble a tattoo needle to the tattoo tube, and then assemble the tube to a tattoo machine, thereby operating the tattoo device to make tattoos.

As shown in FIG. 3, together with FIGS. 1-2, a tattoo tube 50 having a tattoo needle tip equipped with a capillary ink reservoir and a handle in accordance with an embodiment of the present invention is provided. The tattoo needle tip 10 with a capillary ink reservoir is connected with the handle 30 as one unit. The handle 30 comprises a handle body. The handle body comprises a holding grip 31, and a handle neck 32 provided at one end of the holding grip 31. The tattoo needle tip 10 with a capillary ink reservoir is connected with the other end of the holding grip 31 as one unit by the end of the needle tip body 11 away from the needle tip opening 12. The handle body is provided with a guide hole (not shown) that is connected with the tattoo needle passage 100 by in turn perforating the inside of the handle neck 32 and the holding grip 31.

As shown in FIG. 4, together with FIGS. 1-2, a tattoo tube having a tattoo needle tip equipped with a capillary ink reservoir and a handle in accordance with another embodiment of the present invention is provided. The tattoo needle tip 10 with a capillary ink reservoir is demountable to be assembled to the handle 30. The handle 30 comprises a handle body. The handle body comprises a holding grip 31, and a handle neck 32 and a needle tip installation part 33. The handle neck 32 and the needle tip installation part 33 are respectively provided at the two opposite ends of the holding grip 31. The handle body is provided with a guide hole (not shown) that is connected with the needle tip installation part 33 by in turn perforating the inside of the handle neck 32 and the holding grip 31. The tattoo needle tip 10 with a capillary ink reservoir is demountable to be assembled to the needle tip installation part 33. In the present embodiment, the needle tip installation part 33 is provided at the inner side of the opening of a guide hole at one end of the holding grip 31 and the needle tip installation part 33 is provided with a female connector 331. The outside of the end of the needle tip body 11 away from the needle tip opening 12 is provided with a male connector 111 corresponding to the female connector 331. The male connector 111 of the needle tip body 11 is fastened in the female connector 331 to form a demountable quick coupling mechanism, whereby the tattoo needle tip 10 is demountable to be assembled to the handle 30. The needle tip body 11 and the needle tip installation part 33 can also be assembled by coupling of shaft and hole. The structure of the assembled needle tip body 11 and the handle 30 is shown in FIG. 3. The demountable assembling form of the tattoo needle tip 10 and the handle 30 is convenient for a tattooist to change the tattoo needle during making tattoos, thereby being unnecessary to change the handle.

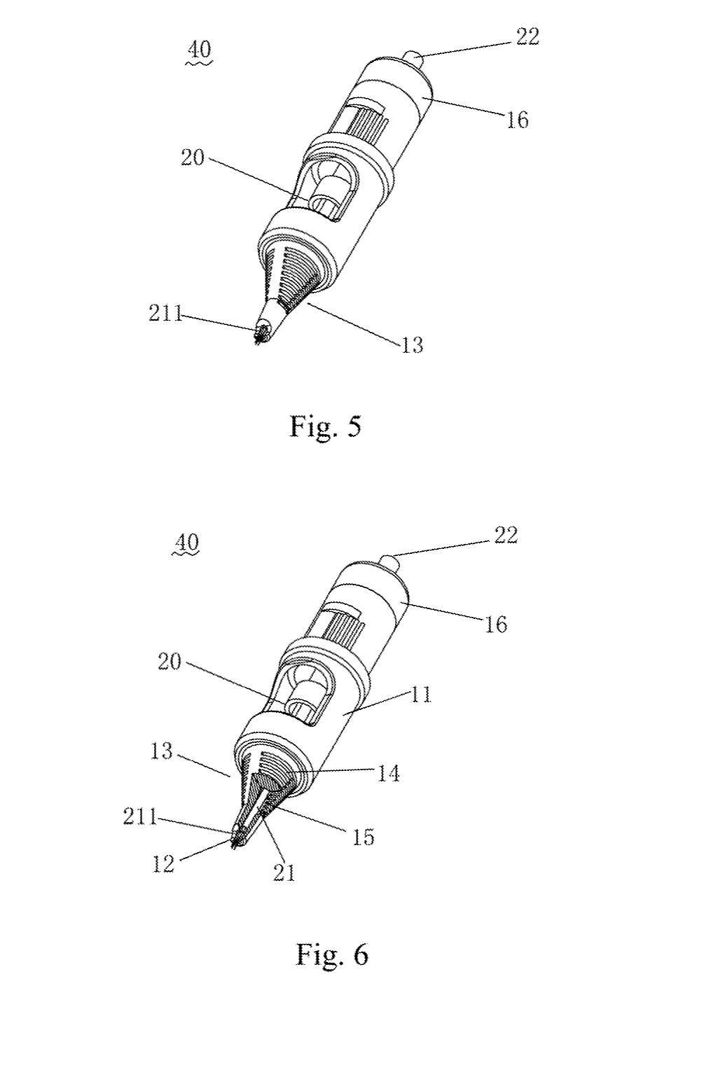

Referring to FIGS. 5-6, together with FIGS. 1-2, the present invention provides an assembly 40 of a tattoo needle tip equipped with a capillary ink reservoir and a tattoo needle. The assembly 40 comprises: a tattoo needle tip and a tattoo needle 20 assembled in the tattoo needle tip.

The tattoo needle tip is configured as the above-mentioned tattoo needle tip 10 with a capillary ink reservoir, which comprises a needle tip body 11. A tattoo needle passage 100 is provided inside the needle tip body 11. A needle tip opening 12 is provided at one end of the needle tip body 11. The needle tip opening 12 and tattoo needle passage 100 are connected. The needle tip body 11 is provided with at least one ink reservoir 13 for containing and storing ink. The ink reservoir 13 is provided with at least one ink chamber 14 with capillary action. At least one ink guiding passage 15 connects with the ink chamber 14 and the needle tip opening 12. The tattoo needle 20 comprises a needle head 21 and a needle bar 22 connecting with the needle head 21. The needle head 21 is contained in the tattoo needle passage 100 of the needle tip body 11. The sharp end 211 of the needle head 21 is located at one end in the tattoo needle passage 100 near the needle tip opening 12. The needle bar 22 protrudes out of the tattoo needle passage 100 from the end of the needle tip body 11 away from the needle tip opening 12, so as to connect the component for driving the tattoo needle 20. The tattoo ink can be contained and stored in the ink chamber 14 of the ink reservoir 13, and can flow through the ink guiding passage 15 to the needle tip opening 12, thereby being dipped by a tattoo needle to make tattoos.

In the present embodiment, the other end of the needle tip body 11 opposite to the needle tip opening 12 is an open end which is further provided with a cover 16 covering thereon. The cover 16 is provided with a hole perforating the tattoo needle passage 100 in the needle tip body 11, for the needle bar 22 of the tattoo needle 20 to protrude therefrom.

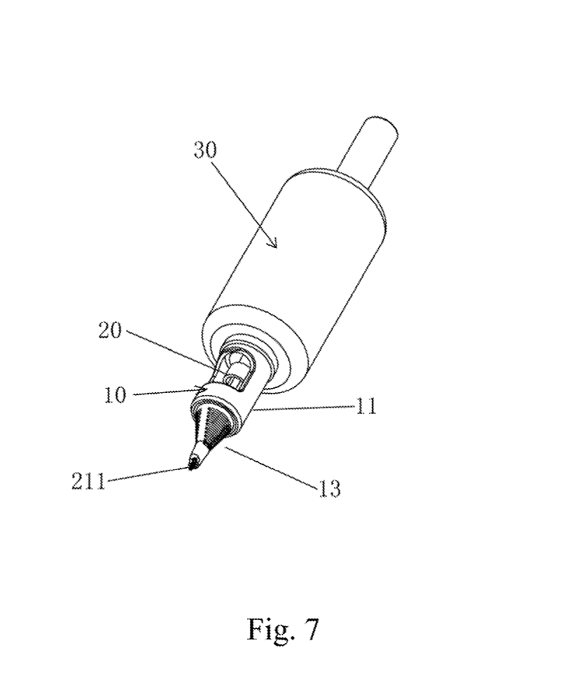

Referring to FIG. 7, together with FIGS. 1-6, while using, the above-mentioned tattoo needle tip 10 with a capillary ink reservoir, tattoo needle 20 and handle 30 are assembled together. The handle 30 is connected with a tattoo machine, and the needle bar 22 of the tattoo needle 20 passes through the through hole of the handle 30 to connect a driving component, thereby forming a tattoo device with the tattoo machine. Driven by the tattoo machine, the tattoo needle 20 processes a high-frequency, linear reciprocating motion in the tattoo needle tip 10. A tattoo artist holds the handle 30 to control the tattoo needle 20 to make tattoos. The ink reservoir 13 of the tattoo needle tip 10 with a capillary ink reservoir is filled with ink. The ink is transported to the needle tip opening 12 via the ink guiding passage 15. While working, the tattoo needle 20 processes a high-frequency, linear reciprocating motion, so, The sharp end 211 dipped with the ink rushes out of the needle tip opening 12 to penetrate the skin, and then The sharp end 211 returns to the needle tip opening 12 to dip the ink to rush out again. While adding the ink, the tattoo needle tip 10 is dipped in a small ink cup, and then the tattoo machine is started for a short time. The tattoo needle 20 will process an up-and-down, reciprocating, high-speed motion, and the ink will quickly pass through the needle tip opening 12 and the ink guiding passage 15 to fill the ink chamber 14 of the ink reservoir 13. While making tattoos, the ink at the needle tip opening 12 is continually dipped by the sharp end 211 of the needle head 21 of the tattoo needle 20 and taken into the skin, and the ink in the ink chamber 14 passes through the ink guiding passage 15 and the tattoo needle passage 100 to flow towards the needle tip opening 12, by the action of capillary force, gravity, and the motion of the tattoo needle 20. The ink dipped by the tattoo needle tip 10 at one time and then stored can be dipped by the sharp end 211 of the tattoo needle for many times, which can last a comparatively long time, thereby preventing the tattoo artist from frequently dipping the ink while making tattoos, whereby the time of tattoo process is saved, and the lines of tattoos is more smooth.

In summary, the present invention provides the tattoo needle tip with an ink reservoir, and utilizes capillary action to store ink in the ink reservoir, thereby having big capacity of ink storage and preventing ink leakage from easily occurring. The ink in the ink reservoir is continuously transported through the ink guiding passage to the needle tip opening. The needle tip continuously dips the ink at the needle tip opening by the reciprocating motion of the tattoo needle. Thus, the ink dipped by the tattoo needle tip at one time can last the tattoo process for a comparatively long time, thereby preventing the tattoo artist from frequently dipping the ink while making tattoos, and saving the time of tattoo process.

Although the present invention has been described in detail with above said embodiments, it is not to limit the scope of the invention. So, all the modifications and changes according to the characteristic and spirit of the present invention are involved in the protected scope of the invention.

What is claimed is:

1. A tattoo needle tip equipped with a capillary ink reservoir comprising: a needle tip body; a tattoo needle passage for mounting a tattoo needle being provided inside the needle tip body; a needle tip opening for the tattoo needle to protrude being provided at one end of the needle tip body, and the needle tip opening and the tattoo needle passage being connected; the needle tip body being provided with at least one ink reservoir for containing and storing ink; the ink reservoir being provided with at least one ink chamber with capillary action; at least one ink guiding passage for connecting the capillary ink chamber with the needle tip opening.

2. The tattoo needle tip equipped with a capillary ink reservoir of claim 1, wherein the ink chamber is a plurality of capillary grooves provided on the ink reservoir.

3. The tattoo needle tip equipped with a capillary ink reservoir of claim 1, wherein the ink reservoir is made from porous materials or fiber materials, and, the capillary ink chamber is pores on the ink reservoir or interstices among fibers.

4. The tattoo needle tip equipped with a capillary ink reservoir of claim 1, wherein the ink reservoir is arranged on the needle tip body as one unit.

5. The tattoo needle tip equipped with a capillary ink reservoir of claim 1, wherein the ink reservoir is demountable to be assembled to the needle tip body; the ink guiding passage is arranged on the needle tip body between the ink reservoir and the tattoo needle passage.

6. The tattoo needle tip equipped with a capillary ink reservoir of claim 1, wherein the ink guiding passage is formed by the tattoo needle passage.

7. A tattoo tube having a tattoo needle tip equipped with a capillary ink reservoir of claim 1 comprising: a handle, and a tattoo needle tip equipped with a capillary ink reservoir comprising: a needle tip body; a tattoo needle passage for mounting a tattoo needle being provided inside the needle tip body; a needle tip opening for the tattoo needle to protrude being provided at one end of the needle tip body, and the needle tip opening and the tattoo needle passage being connected; the needle tip body being provided with at least one ink reservoir for containing and storing ink; the ink reservoir being provided with at least one ink chamber with capillary action; at least one ink guiding passage for connecting the capillary ink chamber with the needle tip opening.

8. The tattoo tube having a tattoo needle tip equipped with a capillary ink reservoir of claim 7, wherein the handle comprises a handle body; the handle body comprises a holding grip, and a handle neck provided at one end of the holding grip; the tattoo needle tip equipped with a capillary ink reservoir is connected with the other end of the holding grip as one unit; the handle body is provided with a guide hole that is connected with the tattoo needle passage by in turn perforating the inside of the handle neck and the holding grip.

9. The tattoo tube having a tattoo needle tip equipped with a capillary ink reservoir of claim 7, wherein the handle comprises a handle body; the handle body comprises a holding grip, a handle neck and a needle tip installation part; the handle neck and the needle tip installation part are respectively provided at the two opposite ends of the holding grip; the handle body is provided with a guide hole that is connected with the needle tip installation part by in turn perforating the inside of the handle neck and the holding grip; the tattoo needle tip equipped with a capillary ink reservoir is demountable to be assembled to the needle tip installation part.

10. The tattoo tube having a tattoo needle tip equipped with a capillary ink reservoir of claim 9, wherein the needle tip installation part is provided at the inner side of the opening of a guide hole at one end of the holding grip, and the needle tip installation part is provided with a female connector; the outside of the end of the needle tip body away from the needle tip opening is provided with a male connector corresponding to the female connector; the male connector of the needle tip body is fastened in the female connector.

11. An assembly of a tattoo needle tip equipped with a capillary ink reservoir of claim 1 and a tattoo needle, comprising a tattoo needle tip equipped with a capillary ink reservoir and a tattoo needle assembled in the tattoo needle tip; the tattoo needle tip equipped with a capillary ink reservoir comprising: a needle tip body; a tattoo needle passage for mounting a tattoo needle being provided inside the needle tip body; a needle tip opening for the tattoo needle to protrude being provided at one end of the needle tip body, and the needle tip opening and the tattoo needle passage being connected; the needle tip body being provided with at least one ink reservoir for containing and storing ink; the ink reservoir being provided with at least one ink chamber with capillary action; at least one ink guiding passage for connecting the capillary ink chamber with the needle tip opening, the tattoo needle being mounted in the tattoo needle passage of the needle tip body, and the sharp end of the needle head of the tattoo needle being located at one end in the tattoo needle passage near the needle tip opening.

12. The assembly of a tattoo needle tip equipped with a capillary ink reservoir and a tattoo needle of claim 11, wherein the tattoo needle comprises a needle head and a needle bar connecting with the needle head; the needle head is contained in the tattoo needle passage of the needle tip body; the needle head is located at one end in the tattoo needle passage near the needle tip opening; the needle bar protrudes out of the needle tip body from the end of the tattoo needle passage away from the needle tip opening, so as to connect a component for driving the tattoo needle.
